(19) United States
(12) Patent Application Publication
(10) Pub. No.: US 2009/0312633 A1
(43) Pub. Date: Dec. 17, 2009

(54) REFERENCE MEANS FOR PRECISION RADIATION TREATMENT

(76) Inventors: Anders Widmark, Umea (SE); Per-Olov Löfroth, Umea (SE)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 12/307,371

(22) PCT Filed: Jul. 4, 2007
(86) PCT No.: PCT/SE07/00661

§ 371 Date: Jan. 5, 2009

(30) Foreign Application Priority Data
Jul. 5, 2006 (SE) 0601472-4

(51) Int. Cl. A61M 25/095; A61B 6/00

(52) U.S. Cl. 600/433

(57) ABSTRACT

The present invention relates to a reference means (1) comprising at least one reference marker (4) which is applicable temporarily or permanently in a patient's body at a specified location relative to a tumor in the body in order thereafter, by means of the reference marker (4), to enable precision aiming of a treatment beam at the tumor. For universal use and easy application of the reference marker, the reference marker (4) is so configured that it is displayable by both magnetic resonance tomography (MRT) and X-raying, preferably CT, in order to determine by MRT and/or X-raying, preferably CT, the location of the reference marker (4) relative to the tumor for subsequent precision aiming of the treatment beam, and the reference marker (4) is applicable, as desired, either temporarily in the body via a catheter (2) which forms part of the reference means (1) or permanently in the body via an insertion means (11) in the form of a catheter.

The present invention relates to a reference means comprising at least one reference marker which is applicable temporarily or permanently in a patient's body at a specified location relative to a tumor in the body in order thereafter, by means of the reference marker, to enable precision aiming of a treatment beam at the tumor.

Diagnostics for target determination and dosage planning in radiation treatment have hitherto been based on X-ray technology, particularly on computer tomography (CT). To ensure the appropriate extent and positioning of the target region (the tumor) intended for radiation treatment, various types of marker technology have been used, particularly in the treatment of tumors in the prostate gland. That technology is based either on permanently implanting X-ray-dense markers in the prostate or on temporarily applying, via a catheter, X-ray-dense markers in the target region and determining the positions of said markers relative to the tumor by X-raying, preferably by CT, with subsequent verification by means of the radiation treatment appliance's imaging system at the time of treatment. In this way it is possible to ensure precision treatment even if the target region is mobile relative to other parts of the patient's anatomy and to any external reference markers tattooed or inked on the patient's skin as sites for radiation treatment fields. The procedure for using reference markers applicable temporarily in the body via a catheter for target determination and dosage planning in radiation treatment is described in more detail in, for example, SE patent specification 9700984-9.

A new diagnostic technique in the form of magnetic resonance tomography (MRT) has been increasingly used for mapping the extent of tumor diseases and has become a routine form of investigation in many such diseases. In many situations, MRT gives better resolution between different tissues than CT, thereby achieving significantly more reliable detection and delineation of a tumor relative to normal tissue around the tumor. MRT of the prostate gives better information than CT about the extent of the gland and can in certain cases show in more detail the extent of a tumor in the prostate gland and more reliably distinguish the prostate from the bottom of the pelvis, the urinary bladder and the rectum. MRT is not yet a generally applied routine in the investigation of localized prostate tumors but the introduction of this technique is being discussed and it is likely to become part of the routines in a near future.

The present invention enhances the potential for being able to use magnetic camera equipment not merely for diagnostic purposes as a supplement to CT but also for dosage planning in radiation treatment, thereby possibly replacing computer tomography equipment.

The need to use marker technology in radiation treatment will therefore continue even if MRT becomes in the future a basic part of diagnostics. However, the metal markers currently used emit insufficient signals to enable them to be detected and their location to be determined with sufficient precision by MRT.

The object of the present invention is therefore to render the reference marker in the means as above usable universally and to configure it in such a way that it can be displayed by both magnetic resonance tomography (MRT) and X-raying, preferably computer tomography (CT), in order to determine by MRT and/or X-raying, preferably CT, the reference marker's position relative to a tumor for subsequent precision aiming of the treatment beam.

With a view to its being not only usable universally but also easy to apply, the reference marker is applicable, as desired, either temporarily in the body via a catheter which forms part of the reference means or permanently in the body via an insertion means in the form of a catheter. In other words, the reference marker according to the invention makes it possible to choose whether it should be applied merely temporarily in the body or whether a more permanent application of the reference marker is desirable.

The above and other features characterizing the invention are described below in more detail with reference to the attached drawings, in which

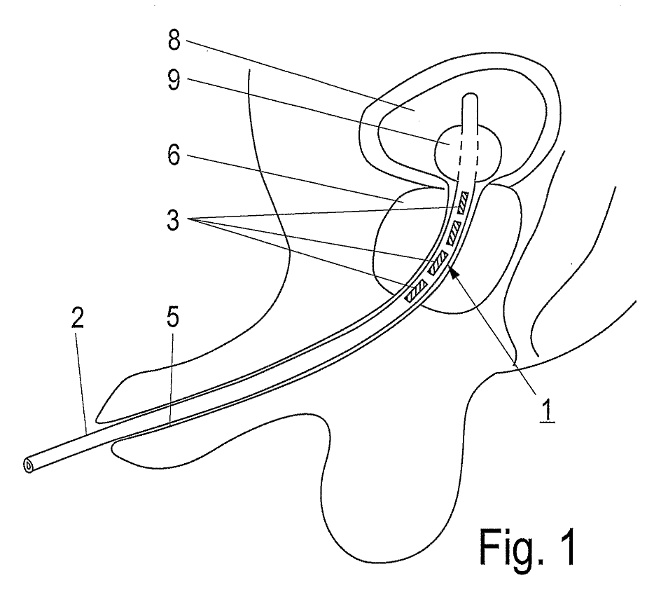

FIG. 1 depicts schematically a reference means not covered by the invention, disposed in an active state with temporary application of reference markers in a patient's body for precision aiming of a treatment beam;

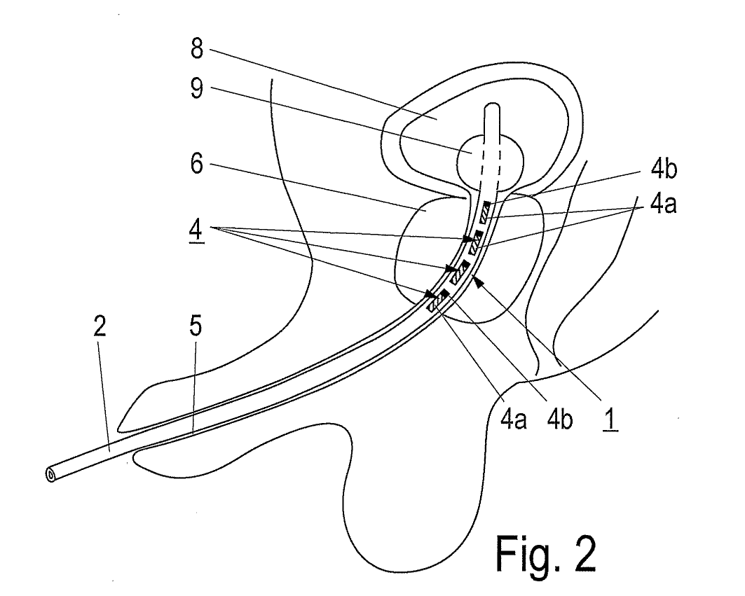

FIG. 2 is a view corresponding to FIG. 1 and depicts schematically a reference means according to the invention in an advantageous embodiment disposed in an active state with temporary application of reference markers;

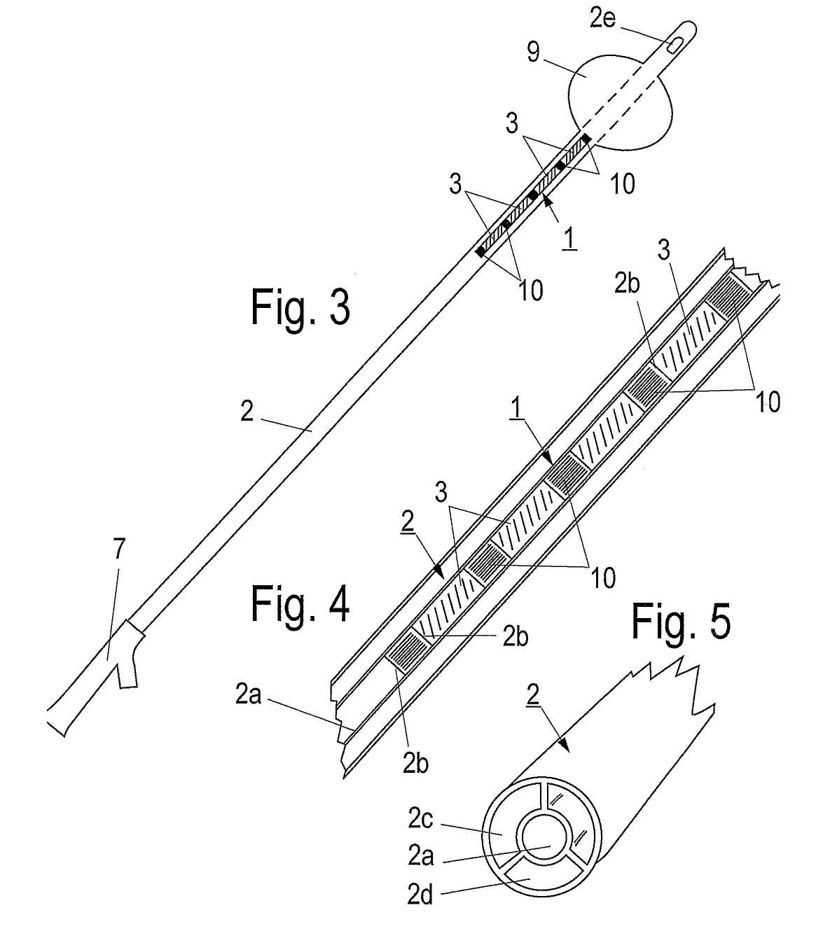

FIG. 3 depicts schematically another reference means not covered by the invention for temporary application of reference markers, but before the reference means is disposed in an active state;

FIG. 4 is an enlarged view of a portion of the reference means according to FIG. 3;

FIG. 5 is an enlarged view of a portion of an example of an embodiment of the catheter which forms part of the reference means;

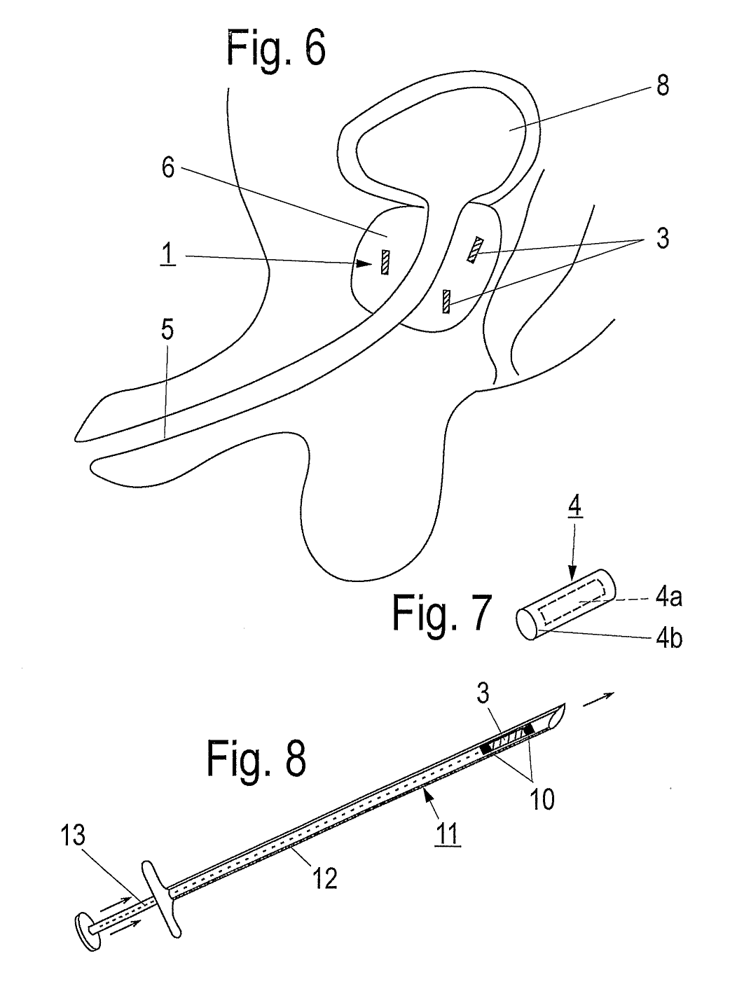

FIG. 6 depicts schematically a reference means not covered by the invention in an active state with permanent application of reference markers in a patient's body for precision aiming of a treatment beam;

FIG. 7 depicts schematically a reference means according to the invention in another advantageous embodiment for permanent application, but before it is disposed in an active state; and

FIG. 8 depicts schematically a further reference means not covered by the invention for permanent application of reference markers, and a means of inserting it.

The reference means 1 depicted in the drawings comprises, according to FIGS. 1 and 2, one or more, in the embodiment depicted four, reference markers 3 and 4 respectively disposed at predetermined distances from one another in the longitudinal direction of a catheter 2, the reference markers 4 being configured according to the present invention. The reference markers 3, 4 may be disposed permanently in the catheter 2 or be disposed insertably therein, e.g. in a duct 2a in the catheter 2 (see FIGS. 4 and 5). The respective reference markers 3, 4 are applicable temporarily in a patient's body through the catheter 2, in FIG. 1 via a patient's urethra 5, at a specified location relative to a tumor in the body, in FIG. 1 a tumor in the prostate gland 6. This is achieved by the catheter 2, which has for example a nozzle element 7 at the opposite end from the insertion end, being inserted in a suitable manner into the body, in FIGS. 1 and 2 far enough into the urethra 5 for the catheter's 2 free end to enter the urinary bladder 8, followed by a fixing means 9, e.g. a balloon means, being expanded inside the urinary bladder in order to fix the catheter relative to the urethra and hence relative to the prostate gland 6 and the tumor therein. The catheter 2 also has with advantage (see FIG. 5) at least one duct, e.g. the duct 2c, for the purpose, via said duct and a control means, e.g. a valve, in the nozzle element 7, of activating the fixing means 9, and at least one further duct, e.g. the duct 2d, for the purpose, via this latter duct and a hole 2e (FIG. 3) at the catheter's insertion end, of providing communication with the urinary bladder 8 from the nozzle element. The fixing of the catheter 2 also fixes the reference markers 3, 4 relative to the prostate gland 6 and the tumor therein. The respective reference markers 3 according to FIG. 1 are so configured as to be displayable by magnetic resonance tomography (MRT), whereas the respective reference markers 4 according to FIG. 2 are so configured as to be displayable by both MRT and X-raying, preferably computer tomography (CT). Each reference marker's location relative to the tumor/the prostate gland is determined by said MRT or by said MRT and/or X-raying, preferably CT. Said MRT or said MRT and/or X-raying, preferably CT, is/are also used for determining the tumor's geometry and planning its treatment relative to the reference markers 3, 4. After, with advantage, initial verification of the mutual positions of the reference markers 3, 4 and of the treatment beam aiming means and any necessary adjustment thereof, a treatment beam can then, on the basis of the reference markers, be aimed with precision at the tumor.

The respective reference markers 3 displayable by MRT may for example take the form of a plastic tube running in the longitudinal direction of the catheter 2. However, the respective reference markers 3 may also take the form of the duct 2a or a section of said plastic tube or duct or any desired element delineating at least one space and made of plastic or similar preferably tissue-compatible material which emits no signals at least in response to MRT. It is in any case essential that the plastic tubes or the ducts or the sections also delineate a space and that this space contains, for example, a liquid or a powder made of material which emits signals in response to MRT. Said space runs along the whole or parts of the length of the catheter 2 and has preferably a length of about 3-10 mm and preferably a diameter of about 1-3 mm.

The aforesaid sections of the plastic tube or the duct 2a in the catheter 2 may be formed, for example, by the plastic tube or the duct being provided with one or more intermediate walls 2b (FIG. 4). These sections may thereafter, according to need or as desired, be filled with the aforesaid signal-emitting material. Between sections with material there may be sections without material, i.e. voids, so that the reference markers 3 constituted by the first-mentioned sections are at a distance from one another.

The respective reference markers 3 displayable by MRT may also take the form of an element made of metal, metal alloy, composite material, some other chemical compound or similar preferably tissue-compatible material which emits signals in response to MRT. As previously indicated, these elements are applied in the catheter 2, possibly in a plastic tube or in the duct 2a in the catheter or in sections of the plastic tube or the duct.

The respective reference markers 4 according to the invention which are displayable by both MRT and X-raying, preferably CT, may, as above, take the form of a plastic tube, duct 2a or section of the plastic tube or the duct running in the longitudinal direction and delineating at least one space, or of some other element delineating at least one space and made of preferably tissue-compatible plastic or similar material which emits signals in response neither to MRT nor to X-raying, preferably CT, and of liquid and/or powder disposed in said space and made of material which emits signals in response to both MRT and X-raying, preferably CT. Said space may here by divided into at least one first portion with material which emits signals in response to MRT and at least one second portion with material which emits signals in response to X-raying, preferably CT.

Accordingly, such a reference marker may also within the scope of the invention take the form of, for example, a plastic tube, duct or section thereof or some other element with a delineated space which is divided by an intermediate wall into two portions containing two different materials, e.g. in liquid form or powder form, or which is undivided and contains instead, for example, a powder in a liquid, where the powder emits signals in response to MRT and the liquid emits signals in response to X-raying, preferably CT, or vice versa.

On the other hand, the respective reference markers 4 displayable by both MRT and X-raying, preferably CT, may also take the form of an element made of preferably tissue-compatible metal, metal alloy, composite material, some other chemical compound or similar material which emits signals in response to both MRT and X-raying, preferably CT. The reference markers 4 may here again be configured with at least one first portion 4a displayable by MRT and at least one second portion displayable by X-raying, preferably CT, i.e. it may comprise, for example, two different metals. According to a preferred embodiment, each such reference marker 4 is provided with at least one core 4a of material which emits signals in response to MRT and an outer layer 4b of material which emits signals in response to X-raying, preferably CT. These elements are likewise applied in the catheter 2, e.g. in a plastic tube or in the duct 2a in the catheter or in sections of the plastic tube or the duct as above, which emit signals in response neither to MRT nor to X-raying, preferably CT.

The reference means may also comprise one or more, according to FIGS. 3 and 4 four, reference markers 3 displayable by MRT, and one or more, in FIGS. 3 and 4 five, reference markers 10 displayable by X-raying, preferably CT, which are located permanently in the catheter 2 relative to the reference markers 3. The location of these last-named reference markers 10 relative to the tumor is therefore determined by said X-raying, preferably CT. After suitable initial verification and any necessary adjustment as above, these reference markers 10 can also be used for precision aiming of the treatment beam.

Exactly like the reference markers 3 and the reference markers 4, the reference markers 10 take the form with advantage of an element made of metal, metal alloy, composite material, some other chemical compound or similar tissue-compatible material, preferably gold or platinum, which emits signals in response to X-raying, preferably CT, or alternatively take the form of an element delineating at least one space, which space contains material which emits signals in response to X-raying, preferably CT.

The reference markers 3 and 10 respectively may be arranged in many different ways relative to one another. Since in the embodiment depicted the reference means 1 comprises a plurality of reference markers 3 displayable by MRT which are disposed in the catheter 2 at predetermined distances from one another, the respective reference markers 10 displayable by X-raying, preferably CT, may with advantage be disposed, as seen in the catheter's longitudinal direction, between two mutually adjacent reference markers 3 which are displayable by MRT. Correspondingly, if the reference means 1 comprises a plurality of reference markers 10 displayable by X-raying, preferably CT, which are disposed in the catheter 2 at predetermined distances from one another, the respective reference markers 3 displayable by MRT may with advantage be disposed, as seen in the catheter's longitudinal direction, between two mutual adjacent reference markers 10 which are displayable by X-raying, preferably CT. Since, as previously indicated, the reference means 1 in the embodiment depicted comprises a plurality of reference markers 10 displayable by X-raying, preferably CT, which are disposed in the catheter 2 at predetermined distances from one another, and also a plurality of reference markers 3 displayable by MRT which are disposed in the catheter 2 at predetermined distances from one another, the reference markers 3, 10 may with advantage be disposed, as seen in the catheter's longitudinal direction, in such a way that alternate reference markers 3 are displayable by MRT and alternate reference markers 10 are displayable by X-raying, preferably CT. As in the aforesaid embodiment with sectioned plastic tubes and ducts, the various sections may for example be filled respectively with material which emits signals in response to MRT and material which emits signals in response to X-raying, preferably CT, each as desired.

In the reference means according to FIGS. 6 to 8 the respective reference markers 3, 4 and 10 are intended to be applied permanently in the patient's body via an insertion means 11. This insertion means 11 may be a needle 12, as in FIG. 8, or a catheter. This needle 12 has at least one movement means 13 for inserting the respective reference markers 3, 4 and 10 into the patient's body. In embodiments with pluralities of reference markers 3, 4 and 10, as in FIGS. 6 and 8, the reference markers are with advantage applicable permanently in the body at chosen distances from one another.

The respective reference markers 3 displayable by MRT may, as in temporary application, take the form of an element delineating at least one space and made of tissue-compatible plastic or similar material which does not emit signals in response to MRT, and of liquid or powder disposed in said space and made of material which emits signals in response to MRT. Alternatively the respective reference markers 3 may take the form of an element made of tissue-compatible metal, metal alloy, composite material, some other chemical compound or similar material which emits signals in response to MRT (FIG. 6).

The respective reference markers 4 according to the invention which are displayable by both MRT and X-raying, preferably CT, may also, as in temporary application, take the form of an element delineating at least one space and made of tissue-compatible plastic or similar material which emits signals in response neither to MRT nor to X-raying, preferably CT, and of liquid and/or powder disposed in said space and made of material which emits signals in response to both MRT and X-raying, preferably CT. Here again said space may be divided into at least one first portion with material which emits signals in response to MRT and at least one second portion which emits signals in response to X-raying, preferably CT. Thus, as previously indicated, the delineated space may be divided into two portions each containing, for example, a liquid and a powder or may comprise a single space with, for example, a powder in a liquid, whereby the powder comprises material which emits signals in response to MRT and the liquid comprises material which emits signals in response to X-raying, preferably CT, or vice versa.

The reference markers 4 according to FIG. 7 take the form of an element made of tissue-compatible metal, metal alloy, composite material, some other chemical compound or similar material which emits signals in response to both MRT and X-raying, preferably CT. As in the last-mentioned case, the respective reference markers 4 are with advantage configured with at least one first portion 4a displayable by MRT and at least one second portion 4b displayable by X-raying, preferably CT, and more particularly with a core 4a which emits signals in response to MRT and an outer layer 4b of tissue-compatible material which emits signals in response to X-raying, preferably CT.

The reference means 1 may finally, as in the embodiment according to FIGS. 3 and 4, comprise at least one, according to FIG. 8 only one, reference marker 3 displayable by MRT and one or more, in FIG. 8 two, reference markers 10 displayable by X-raying, preferably CT, which are applicable permanently in the body relative to the reference markers 3.

Here again the reference markers 10 take with advantage the form of an element made of metal, metal alloy, composite material, some other chemical compound or similar material, preferably gold or platinum, which emits signals in response to X-raying, preferably CT, but may here likewise comprise respective elements delineating at least one space, as the reference markers 3, whereby said space contains material which emit signals in response to X-raying, preferably CT.

The reference markers 3 and 10 respectively may here likewise be arranged in many different ways relative to one another. Thus, if the reference means 1 comprises a plurality of reference markers 10 displayable by X-raying, preferably CT, which are applicable permanently in the body at chosen distances from one another, the respective reference markers 3 displayable by MRT may be permanently applicable in the body relative to, e.g. between, two mutually adjacent reference markers 10 which are displayable by X-raying, preferably CT (see FIG. 8).

Correspondingly, if the reference means 1 comprises a plurality of reference markers 3 displayable by MRT which are applicable permanently in the body at chosen distances from one another, the respective reference markers 10 displayable by X-raying, preferably CT, may be applicable permanently in the body relative to, e.g. between, two mutually adjacent reference markers 3 which are displayable by MRT.

Even if the reference means 1 comprises a plurality of reference markers 3 so configured that they are displayable by MRT and a plurality of said further reference markers 10 so configured that they are displayable by X-raying, preferably CT, the reference markers 3, 10 are with advantage applicable permanently in the body at chosen distances in such a way that alternate reference markers 3 are displayable by MRT and alternate reference markers 10 are displayable by X-raying, preferably CT.

The foregoing makes it clear that the reference means according to the present invention comprises reference markers 4 which are displayable by various technologies individually or in combination and are also applicable in the body temporarily or permanently as desired.

It is also obvious to one skilled in the art that, in addition to the foregoing, the present invention may be modified and altered within the scopes of the claims set out below without departing from the idea and object of the invention. Thus, for example, the reference markers 4 may be configured in further ways other than those indicated above, e.g. the plastic tubes may comprise any suitable material other than plastic which emits signals in response neither to MRT nor to X-raying, preferably CT, and may contain materials or substances other than liquid and/or powder which emit signals in response to both MRT and X-raying, preferably CT.

1: A reference means (1) comprising at least one reference marker (4) which is applicable temporarily or permanently in a patient's body at a specified location relative to a tumor in the body in order thereafter, by means of the reference marker (4), to enable precision aiming of a treatment beam at the tumor, wherein:
the reference marker (4) is so configured that it is displayable by both magnetic resonance tomography (MRT) and X-raying, preferably computer tomography (CT) in order to determine by MRT and/or X-raying, preferably CT, the location of the reference marker (4) relative to the tumor for subsequent precision aiming of the treatment beam, and
the reference marker (4) is applicable, as desired, either temporarily in the body via a catheter (2) which forms part of the reference means (1) or permanently in the body via an insertion means (11) in the form of a catheter.

2: A reference means (1) according to claim 1, wherein the reference marker (4) takes the form of an element delineating at least one space and made of tissue-compatible plastic or similar material which emits signals in response neither to MT nor to X-raying, preferably CT, and of liquid and/or powder disposed in said space and made of material which emits signals in response to both MRT and X-raying, preferably CT.

3: A reference means (1) according to claim 2, wherein said space is divided into at least one first portion (4a) with material which emits signals in response to MRT and at least one second portion (4b) with material which emits signals in response to X-raying, preferably CT.

4: A reference means (1) according to claim 1, wherein the reference marker (4) takes the form of an element made of tissue-compatible metal, metal alloy, composite material, some other chemical compound or similar material which emits signals in response to both MRT and X-raying, preferably CT.

5: A reference means (1) according to claim 4, wherein said element (4) comprises at least one first portion (4a) made of tissue-compatible material which emits signals in response to MRT and at least one second portion (4b) made of tissue-compatible material which emits signals in response to X-raying, preferably CT.

6: A reference means (1) according to wherein said element (4) comprises at least one core (4a) made of material which emits signals in response to MRT and an outer layer (4b) made of tissue-compatible material which emits signals in response to X-raying, preferably CT.

7: A reference means (1) according to claim 1, wherein the reference means (1) comprises a plurality of reference markers (4) which are applicable, as desired, either temporarily or permanently in the body at chosen distances from one another.
